(19) United States
(12) Patent Application Publication
(10) Pub. No.: US 2007/0293939 A1
(43) Pub. Date: Dec. 20, 2007

(54) FATIGUE RESISTANT ENDOPROSTHESES

(75) Inventors: Sanjay Shrivastava, Mountain View, CA (US); Kevin Kang, San Jose, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Laboratories, Abbott Park, IL (US)

(21) Appl. No.: 11/748,214

(22) Filed: May 14, 2007

Related U.S. Application Data
(60) Provisional application No. 60/800,332, filed on May 15, 2006.

(51) Int. Cl. A61F 2/06

(52) U.S. Cl. 623/1.16

(57) ABSTRACT

A superelastic endoprosthesis can have improved fatigue resistance, and improved resistance to crack formation by being configuring to have an austenitic finish temperature from about 5 degrees Celsius to about 35 degrees Celsius, a stress-strain curve having an upper plateau stress from about 40 ksi to about 80 ksi, and a lower plateau stress from about 5 ksi to about 50 ksi. Such an endoprosthesis may be fabricated by heating at least a portion of the endoprosthetic body in a fluid, such as air, salt bath, or fluidized sand, having a temperature from about 400 degrees Celsius to about 600 degrees Celsius for at least about 30 seconds. Additionally, only portions of an endoprosthesis may selectively be subjected to the heating parameters of the present invention such that the endoprosthesis exhibits an increased radial stiffness and an increased flexibility in the longitudinal direction.

### CROSS-REFERENCE TO RELATED APPLICATIONS

This U.S. patent application claims benefit of having U.S. provisional patent application Ser. No. 60/800,332, filed on May 15, 2006, entitled “FATIGUE AND FRACTURE RESISTANT STENTS THROUGH ALTERATION OF MECHANICAL PROPERTIES OF NITINOL,” and having Sanjay Shrivastava, and Kevin Kang as inventors, which U.S. provisional patent application is incorporated herein in its entirety by specific reference.

### BACKGROUND

I. Technology Field

The present invention relates to fatigue resistant superelastic endoprostheses. More particularly, the present invention relates to endoprostheses that have modulated austenitic finish temperatures and modulated plateau stresses. The present invention also relates to fatigue resistant superelastic endoprostheses having anisotropic stress measurements in the radial and longitudinal directions.

II. The Related Technology

Superelastic endoprostheses are subjected to multiple stresses and strains after their insertion into a body lumen. Due to the constant stresses and strains placed upon an endoprosthesis, microcracks may eventually form on the surface or within a superelastic endoprosthesis leading to structural failure of the endoprosthesis. Increasing the overall strength of a superelastic endoprosthesis can lead to an endoprosthesis that is too rigid and inflexible and thus may damage the vessel or other body lumen in which it is placed.

Superelastic materials possess unique characteristics that are particularly useful in endoprosthetic applications. If a piece of a shape memory alloy, such as nitinol, is mechanically stretched, compressed, bent, or twisted in its martensitic phase, it will return to its original configuration upon heating. Typically, thermal shape memory is used wherein the shape of the shape memory alloy is set to the recovery state by deforming the austenitic material at high temperature, cooling the material to a martensitic state and deforming the martensitic material into a given shape, then heating the martensitic alloy until it obtains the austenitic temperature and reforms its shape that was set during the high temperature set; the recovery state.

The transformation between austenite and martensite is reversible but the temperature at which it occurs is different whether the shape memory alloy is being cooled or heated. This difference is referred to as the hysteresis cycle. This cycle is characterized by four different temperatures: Aₛ (Austenite Start), A_{f} (Austenite Finish), Mₛ (Martensite Start), and M_{f} (Martensite Finish). A martensitic shape memory alloy will begin to transform to austenite when its temperature reaches Aₛ and will be fully austenitic when the temperature reaches A_{f}. Reversely, upon cooling, martensite will start to appear when the temperature reaches Mₛ and the transformation will be complete when the temperature drops below M_{f}. A number of parameters including alloy composition and thermo-mechanical history can affect the transformation temperatures and can be adjusted for specific applications.

Although the chemical composition is identical, an austenitic form of an alloy and a martensitic form of an alloy have different crystal structures that impart different physical characteristics to the alloy. The martensite crystalline forms upon cooling from the high temperature austenitic phase. The malleable martensitic form of an alloy can be easily deformed and if not constrained, will freely recover upon heating to its original, much stronger austenite phase. In theory, this cycle can be repeated indefinitely. However, micro-cracks begin to form through environmental stresses that the shape memory alloy is subjected to. These micro-cracks eventually lead to a traumatic structural failure within the shape memory alloy.

Various endoprostheses incorporating the use of shape memory elements have been known for a number of years. Endoprostheses adopting the use of shape memory elements, rely on the unique structural properties of shape memory alloys in order to achieve their desired effects. The shape memory alloys that are used in the stents retain their new shape when cooled to the martensitic state and thereafter deformed, however, these same shape memory alloys will recover their original shape when warmed to the austenitic state.

Therefore, it would be advantageous to have superelastic endoprostheses that are resistant to fatigue and crack formation. Also, it would be advantageous to have superelastic endoprostheses that have an increased austenitic final temperature, and a modulated stress-strain curve. Additionally, it would be advantageous to have a superelastic endoprosthesis that is stronger in the radial direction, but also more flexible in the longitudinal direction such that the endoprosthesis would be preferentially stiffened where it contacts a lesion inside of a lumen, but be flexible enough in the longitudinal direction to reach the point within the lumen where the lesion exists without damaging the vessel.

### BRIEF SUMMARY

The present invention generally relates to superelastic endoprostheses having increased fatigue resistance and increased resistance to the formation of cracks. In particular, the present invention relates to methods for selectively increasing the austenitic finish temperature of superelastic endoprostheses as well as to methods for selectively increasing the austenitic finish temperature of particular portions of superelastic endoprostheses. The methods of the present invention include heating cycles designed to impart particular stress plateaus and austenitic finish temperatures upon a superelastic endoprosthesis. The entire endoprosthesis may be heated to increase the austenitic finish temperature or only very narrow portions or pieces of the endoprosthesis may be heated to impart an increased austenitic temperature thereto. The endoprostheses of the present invention may be selectively stiffened in the radial direction as well as having increased flexibility in the longitudinal direction through selectively applying the heating methods of the present invention to particular portions of superelastic endoprostheses. The superelastic endoprostheses may be adapted to be implanted in a body lumen, such as carotid arteries, coronary arteries, peripheral arteries, veins, and/or other vessels or body lumens.

In one embodiment, the superelastic endoprosthesis of the present invention has improved fatigue resistance. The endoprosthesis includes a body of superelastic metal where at least a portion of the superelastic metal is characterized by having an austenitic finish temperature from about 5 degrees Celsius to about 35 degrees Celsius. The superelastic endoprosthesis is characterized to have a stress-strain curve having an upper plateau stress from about 40 ksi to about 80 ksi and a lower plateau stress from about 5 ksi to about 50 ksi. In one embodiment, the superelastic endoprosthesis can be characterized to have an austenitic finish temperature from about 15 degrees Celsius to about 20 degrees Celsius, an upper plateau loading stress from about 60 ksi to about 80 ksi, and a lower plateau unloading stress from about 20 ksi to about 40 ksi. In one embodiment, the superelastic endoprosthesis can be characterized to have an austenitic finish temperature from about 30 degrees Celsius to about 37 degrees Celsius, an upper plateau loading stress from about 40 ksi to about 80 ksi, and a lower plateau unloading stress from about 5 ksi to about 50 ksi.

In one embodiment, the superelastic metal of the endoprosthesis of the present invention may be made out of a nickel-titanium alloy such as nitinol. Nitinol may vary in its composition of nickel and titanium, but is nearly binary in its composition. The endoprosthetic body may be in the form of a stent having substantially annular elements connected together by connectors; however; other well known configurations can be used.

In one embodiment, the entire body, a majority of the body, a minor portion, or only the connector or annular portions of the superelastic endoprosthesis may have the superelastic characteristics as described herein. The endoprosthesis may be heated according to the parameters of the present invention such that the entire endoprosthesis may exhibit the characteristics of having an increased austenitic final temperature, lower loading and unloading plateau stresses, as well as having increased resistance to crack formation and an increased resistance to fatigue.

In one embodiment, an endoprosthetic body having the superelastic metal characteristics as listed above may be fabricated by heating at least a portion of the endoprosthetic body in a fluid, such as air, salt bath, or fluidized sand, having a temperature from about 400 degrees Celsius to about 600 degrees Celsius for at least about 30 seconds. Additionally, only portions of an endoprosthesis may selectively be subjected to the heating parameters of the present invention such that the endoprosthesis exhibits an increased radial stiffness and an increased flexibility in the longitudinal direction.

In one embodiment, the superelastic endoprosthesis may be fabricated through using the method of point heating a portion of the superelastic metal. That portion may include the substantially annular elements and/or connectors. The point heating can come from using a narrowly focused laser, plasma, ion beam, photo beam, or electron beam to point heat a portion of the superelastic endoprosthesis to a temperature range and period of time as disclosed above.

In one embodiment, the superelastic endoprosthesis is selectively configured to have a particular austenitic temperature in the range from about 5 to about 35 degrees Celsius, a particular upper plateau stress in the range of about 40 to about 80 ksi and a particular lower plateau stress in the range of about 5 to about 50 ksi by heating the entire body, a majority of the body, or only the connector or annular portions of the superelastic endoprosthetic at a particular temperature in the range from about 400 degrees Celsius to about 600 degrees Celsius for a period of time of at least about 30 seconds.

In one embodiment, after the superelastic endoprosthesis is heat set to its final diameter, it is selectively configured to have a particular austenitic temperature in the range from about 5 to about 35 degrees Celsius, a particular upper plateau stress in the range of about 40 to about 80 ksi and a particular lower plateau stress in the range of about 5 to about 50 ksi by applying a heating cycle at least once to the entire body, a majority of the body, or only the connector or annular portions of the superelastic endoprosthetic at a particular temperature in the range from about 350 degrees Celsius to about 550 degrees Celsius for a period of time of at least about 30 seconds to about 30 minutes.

In one embodiment, the superelastic endoprosthesis having improved fatigue resistance may be fabricated to selectively impart anisotropism between the longitudinal stiffness and the radial stiffness. The superelastic endoprosthesis may be configured to have a longitudinal stiffness that is more flexible compared to the radial stiffness. This anisotropism is imparted upon the superelastic endoprosthesis through selectively point heating a portion of the superelastic endoprosthesis, including the substantially annular elements or the connectors. The selective heating of the substantially annular elements or the connectors is achieved by using a narrowly focused laser, plasma, ion beam, photo beam, or electron beam to heat the endoprosthesis portion to a temperature in a range from about 400 degrees Celsius to about 600 degrees Celsius for a period of time of at least about 30 seconds.

### BRIEF DESCRIPTION OF THE DRAWINGS

To further clarify the above and other advantages and features of the present invention, a more particular description of the invention will be rendered by reference to specific embodiments thereof that are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a stress-strain hysteresis curve for a nickel-titanium superelastic alloy.

FIG. 2 is a bar graph depicting different upper stress plateaus, in ksi (1 ksi=1000 psi), of endoprostheses having different austenitic finish temperatures.

FIG. 3 is a bar graph depicting different lower stress plateaus, in ksi, of the different stents of FIG. 2.

FIG. 4 is a bar graph depicting the percent distention of the endoprostheses of FIG. 2 before they fracture.

### DETAILED DESCRIPTION OF SELECTED EMBODIMENTS

The present invention generally relates to superelastic endoprostheses having increased fatigue resistance and increased resistance to the formation of cracks. In particular, the present invention relates to methods for selectively increasing the austenitic finish temperature of superelastic endoprostheses as well as to methods for selectively increasing the austenitic finish temperature of particular portions of superelastic endoprostheses. The methods of the present invention include at least one heating cycle designed to impart particular upper and lower stress plateaus and austenitic finish temperatures upon a superelastic endoprosthesis. Additionally, endoprostheses of the present invention may be selectively stiffened in the radial direction as well as having increased flexibility in the longitudinal direction through selectively applying the heating methods of the present invention to particular portions of superelastic endoprostheses. The superelastic endoprostheses may be adapted to be implanted in a body lumen, such as carotid arteries, coronary arteries, peripheral arteries, veins, and/or other vessels. It is recognized that the present invention is not limited to superelastic endoprostheses and may be used in various other medical devices (e.g., guidewires) and components thereof where the same principles are applicable.

I. Introduction

Superelastic endoprostheses are known in the art, but a problem with the use of existing endoprostheses employing shape memory alloys (e.g., superelastic alloys) is that through use, the superelastic alloys form stress fractures and can break while inside of the patient. Microfractures can be created while the superelastic alloy is being shaped into the appropriate diameter through subsequent rounds of heating, cooling, and expansion on mandrels, for example, in order to obtain the proper diameter for the superelastic alloy. Additionally, microfractures leading to failure of an intravascular endoprosthesis can occur due to the stress-strain cycles that the endoprosthesis is subjected to during deployment into a body lumen and while inside the body lumen. Therefore, one embodiment of the present invention includes superelastic endoprostheses that are resistant to fatigue and crack formation.

Additionally, one embodiment of the present invention includes a superelastic endoprosthesis that is stronger in the radial direction, but also more flexible in the longitudinal direction. Such an endoprosthesis can be preferentially stiffened, and therefore strengthened, where it contacts a lesion inside of a lumen, but be flexible enough in the longitudinal direction to facilitate placement within the lumen where the lesion exists without damaging the vessel.

Nitinol is a superelastic alloy of nickel and titanium possessing shape memory. The shape memory characteristic of nitinol is a result of metallurgical phase transformations. Depending on its temperature, the structural properties of nitinol enable it to function in two different states. At the lower temperature range, below a specified transition temperature, nitinol becomes more flexible and malleable and is said to be in the martensitic state. However, when heated above the specified transition temperature, nitinol transforms into its predetermined heat-set shape in the austenitic state.

In one embodiment, the superelastic endoprosthesis can include a superelastic alloy, such as a nickel-titanium composite (e.g., nitinol). Optionally, the superelastic alloy can include a ternary element selected from the group of elements consisting of palladium, platinum, chromium, iron, cobalt, vanadium, manganese, boron, copper, aluminum, tungsten, tantalum, or zirconium.

Within a superelastic endoprosthesis, the transformation between austenite and martensite is reversible but the temperature at which it occurs is different whether the shape memory alloy is being cooled or heated. This difference is referred to as the hysteresis cycle. This cycle is characterized by four, different temperatures: Aₛ (Austenite Start), A_{f} (Austenite Finish), Mₛ (Martensite Start, and M_{f} (Martensite Finish). A martensitic shape memory alloy will begin to transform to austenite when its temperature reaches Aₛ and will be fully austenitic when the temperature reaches A_{f}. Reversely, upon cooling, martensite will start to appear when the temperature reaches Mₛ and the transformation will be complete when the temperature drops below M_{f}. A number of parameters including alloy composition and thermo-mechanical history can affect the transformation temperatures and can be adjusted for specific applications.

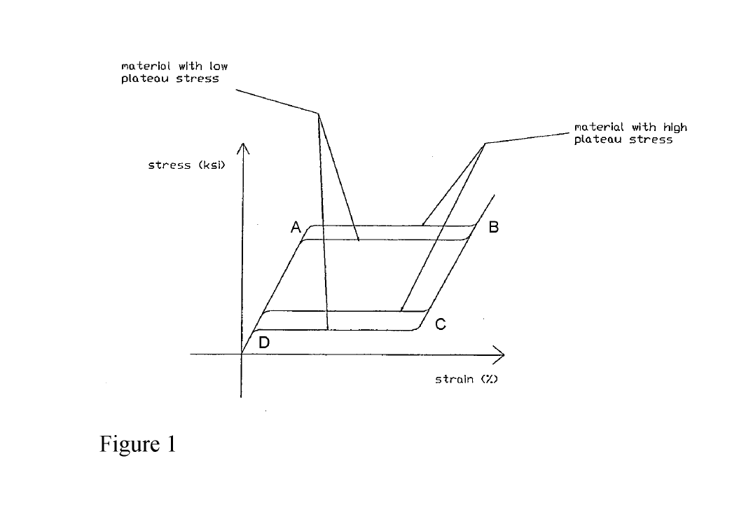

FIG. 1 depicts a stress-strain hysteresis curve for a binary nickel-titanium alloy demonstrating the loading and unloading of the metal alloy. A more detailed discussion of nickel-titanium behavior may be found in T. W. Duerig, A. R. Pelton, “Ti—Ni Shape Memory Alloys, Materials Properties Handbook Titanium Alloys,” pp. 1035-1048, ASM International (1994), the contents of which are incorporated herein by reference. Additional industry literature by D. E. Hodgson, J. W. Brown, “Using Nitinol Alloys,” pp. 1-38, Shape Memory Applications, Inc. (2000), provides an overview of the phenomenon of the shape memory effect exhibited by nitinol, the contents of which are incorporated herein by reference. With regard to the use of nitinol for stent applications, particularly for guidewire applications, the article by A. R. Pelton et al., “Optimization of Processing and Properties of Medical Grade Nitinol Wire,” pp. 107-118, Minimally Invasive Therapy & Allied Technologies (2000), provides a thorough discussion in this area, the contents of which are incorporated herein by reference.

As illustrated in FIG. 1, the hysteresis curve is also generally known as a superelastic curve, which is characterized by areas of nearly constant stress during loading and unloading of the metal alloy. The line segment AB, as depicted in FIG. 1, is the constant loading stress and is referred to as the loading plateau stress while the line segment CD is the constant unloading stress and is referred to as the unloading plateau stress.

The stress-strain hysteresis curve, as depicted in FIG. 1, is for a nickel-titanium alloy tested above its A_{f} but below its martensitic deformation temperature (M_{d}), in its superelastic range. The austenite finish temperature A_{f} is the temperature at which the nickel-titanium alloy completely converts to austenite. The onset of superelasticity occurs in the narrow temperature range just above A_{f}. At M_{d}, nitinol behaves like a non-superelastic metal, exhibiting a small linear elastic range.

Heat is released and absorbed during the martensitic (exothermic) and austenitic (endothermic) transformations. However, the simplest and perhaps most useful method of measuring A_{f} is through the free recovery technique. Measurement through the free recovery technique requires the application of three basic steps which collectively simulate a shape-memory cycle. These specific steps are set forth in the above mentioned article entitled, “Optimization of Processing and Properties of Medical Grade Nitinol Wire,” the contents of which are incorporated herein by reference.

Microfractures can also form when an intraluminal endoprosthesis, for example, is subjected to multiple stress-strain cycles from its surrounding environment. For example, an intraluminal endoprosthesis may be placed within the superficial femoral artery of a patient. Every time that the patient moves, stresses and strains are imparted upon the superficial femoral artery and therefore upon the intraluminal endoprosthesis disposed therein. Microfractures can readily form, and may propagate throughout the structure and cause a catastrophic failure within the superelastic material. In the case of an endoprosthesis, such as a stent, failure of the stent can be catastrophic causing stenosis within a vessel that leads to thrombosis, stroke, or heart attack. Thus, the fatigue resistance of a superelastic endoprosthesis may be increased through using methods to raise the austenitic finish temperature of the superelastic material from which the endoprosthesis is manufactured from.

Superelastic metals and alloys are more resistant to deformation while being subjected to stress and strain compared to non-superelastic metals. Superelastic metal alloys, such as nitinol, have recoverable elastic strains up to 8% at temperatures slightly above their austenitic finish temperature. Other metals such as a stainless steel, can usually recover from strains of only about 0.2% before becoming permanently deformed. This increased resistance to deformation over normal metals allows superelastic materials to be used in applications in which a normal metal would be permanently deformed. The increased resistance to deformation of superelastic metals is especially useful in endoprostheses. Endoprostheses composed of superelastic metals and alloys can be disposed within a body lumen and undergo enormous strains without deforming their prosthetic shape.

The resistance of superelastic metal to deformation is due in part to the transition between the two different crystalline states of the metal. The transition between the crystalline states requires energy, is endothermic, from Aₛ to A_{f}, and releases energy, is exothermic, in the Mₛ to M_{f} transition. The transition of the superelastic metal from one crystalline state to the other is therefore characterized on a stress-strain curve as a plateau, while energy is imparted into or taken away from the superelastic metal. There are two stress plateaus, an upper stress plateau, sometimes referred to as a loading plateau, and a lower stress plateau, sometimes referred to as an unloading plateau.

II. Endoprostheses Having Improved Fatigue Resistance

In one embodiment, superelastic endoprostheses of the present invention are configured to have increased austenitic finish temperatures and increased resistance to fatigue and crack formation. In one embodiment, superelastic endoprostheses are manufactured to have an anisotropism imparted upon the radial and longitudinal stress measurements.

Increasing fatigue resistance in endoprosthesis of increasingly small inner diameters requires superelastic endoprosthetic materials having altered mechanical properties. The present invention of increasing fatigue resistance in superelastic materials by altering the mechanical properties of the superelastic metal from which the endoprosthesis is composed through configured heat applications, addresses the need for increasing fatigue resistance in endoprostheses.

Intraluminal endoprostheses can be configured such that the superelastic endoprosthesis is available in the deformable martensitic state upon its introduction into a living tissue and then transforms into the austenitic state upon warming to the temperature of the surrounding tissue. In the martensitic form, the superelastic endoprosthesis is substantially compact and thus easier to place inside of a vessel.

In a superelastic endoprosthesis which has been placed within a body lumen, the loading stress is created by the forces pushing in on the lumen/endoprosthesis. A high loading plateau (upper plateau stress) is equal to high crush resistance of the stent. Whereas an unloading plateau (lower plateau stress) correlates to force pushing against the lumen wall. A lower unloading plateau is equal to a gentler force pushing on the lumen wall, thus helping to prevent damage to the vessel or other body lumen wall. Superelastic metals and alloys having a higher austenitic finish temperature generally have upper and lower stress plateau levels that are less than those in superelastic metals and alloys having a lower austenitic finish temperature.

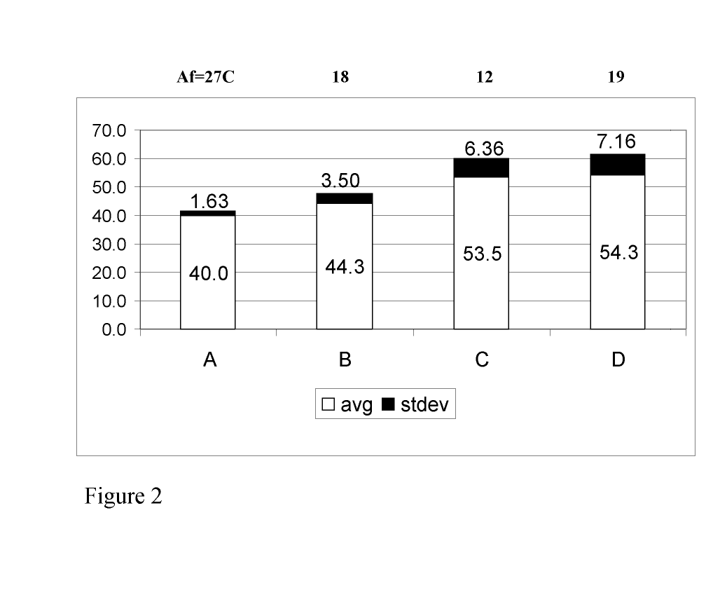

It is established in the art that when the austenitic finish temperature of a superelastic material is configured to be higher, the upper and lower plateau stresses decrease. Surprisingly, when the austenitic finish temperature of various superelastic endoprosthesis were measured against the fatigue resistance of those endoprostheses, an increased austenitic temperature unexpectedly raised the fatigue resistance of the superelastic endoprosthesis. As exemplified in FIGS. 2 through 4, an increase in the austenitic finish temperature of a superelastic metal decreases the upper and lower plateau stresses and increases the fatigue resistance of the superelastic metal.

FIG. 2 is a bar graph depicting different upper stress plateaus of the different stents having different austenitic finish temperatures. The stress plateaus are measured in ksi where one ksi is equal to 1000 psi, pounds per square inch. There is a relationship between the increase in austenitic finish temperature and the decrease in the upper plateau stress. Having plateau stresses that are lower is beneficial to an endoprosthesis because the endoprosthesis does not push out on the luminal wall in which it is inserted with as much force and therefore, the chance of damaging the luminal wall is less than an endoprosthesis having greater plateau stresses.

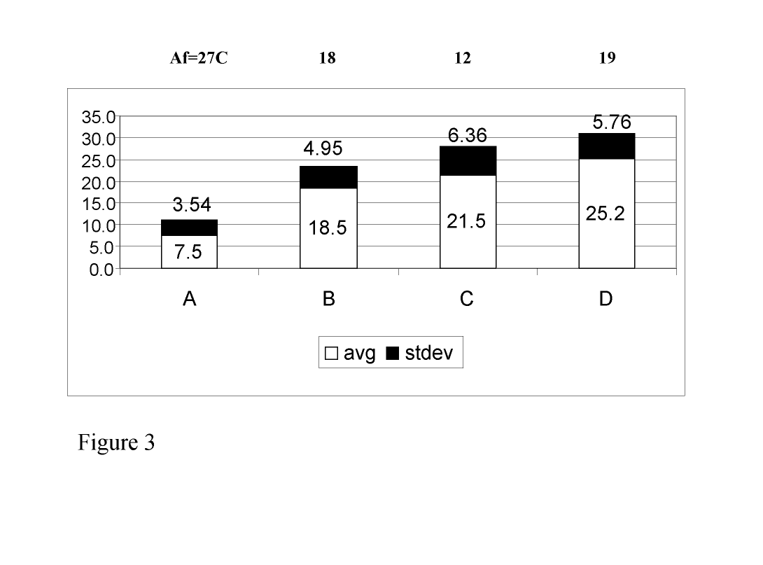

FIG. 3 is a bar graph depicting different lower stress plateaus of the different stents of FIG. 2. The stress plateaus are measure in ksi. In general, the lower stress plateaus decrease with increasing austenitic finish temperature. Therefore, when looking at the data presented in FIGS. 2 and 3, when the austenitic finish temperature of a superelastic metal is increased, both the upper, loading stress plateau and the lower, unloading stress plateau are generally lower.

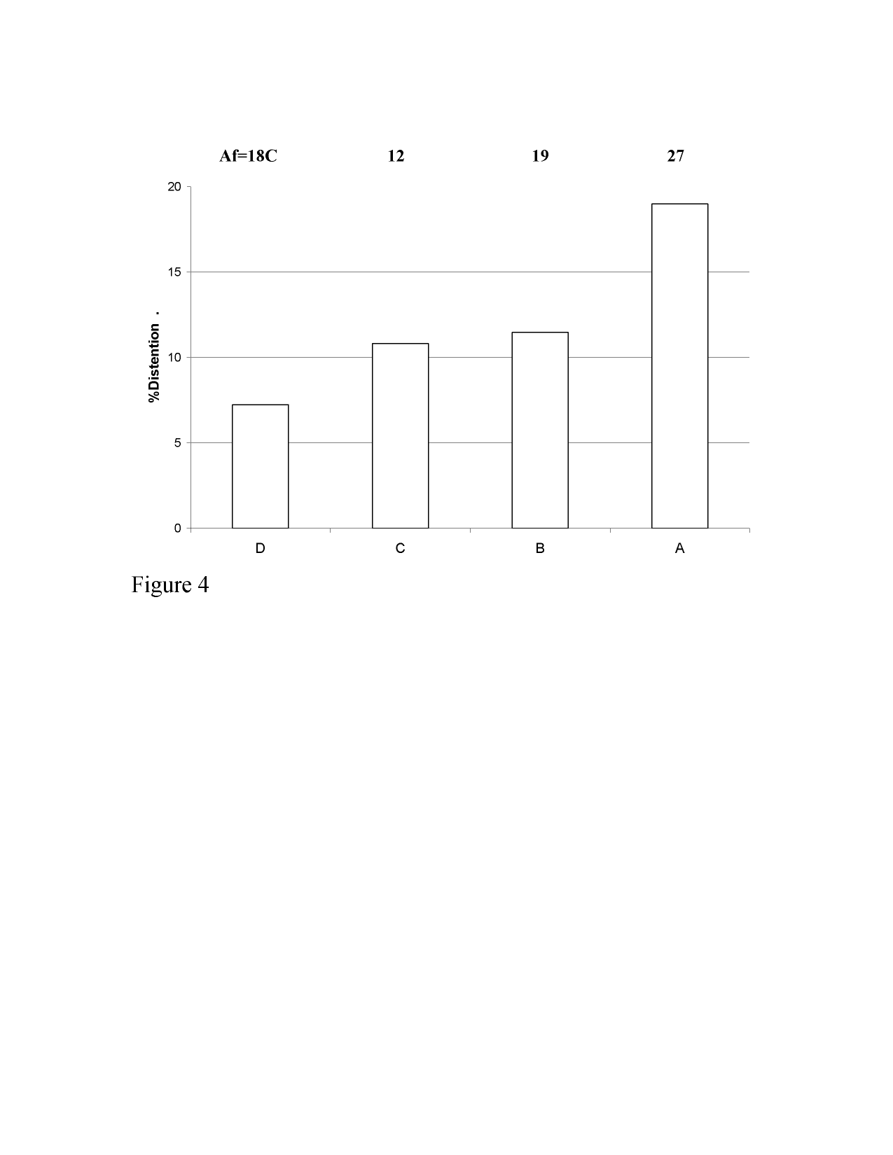

FIG. 4 is a bar graph depicting the percent of distention of the endoprostheses of FIG. 2 before they fracture. FIG. 4 depicts that the general trend amongst the endoprostheses tested is that with increasing austenitic finish temperature, there is an increase in fatigue resistance and resistance to cracking and failure of the endoprosthesis structure.

In one embodiment, superelastic endoprostheses are configured to have an austenitic finish temperature that imparts increased fatigue resistance and increased resistance to the formation of cracks. Such an endoprosthesis can be configured to have an austenitic finish temperature of from about 5 degrees Celsius to about 35 degrees Celsius. In a preferred embodiment, the austenitic finish temperature is from about 10 degrees Celsius to about 30 degrees Celsius. In a more preferred embodiment, the austenitic finish temperature is from about 15 degrees Celsius to about 20 degrees Celsius. It can be advantageous to have an austenitic finish temperature from about 30 degrees Celsius to about 37 degrees Celsius. It can be additionally advantageous to have an austenitic finish temperature from about 31 degrees Celsius to about 36 degrees Celsius. It can be even more advantageous to have an austenitic finish temperature from about 32 degrees Celsius to about 35 degrees Celsius.

In one embodiment, superelastic endoprostheses are configured to have an upper stress plateau that imparts increased fatigue resistance and increased resistance to the formation of cracks. Such an endoprosthesis can be configured to have an upper stress plateau from about 40 ksi to about 80 ksi. In a preferred embodiment, the upper stress plateau is from about 50 ksi to about 70 ksi. In a more preferred embodiment, the upper stress plateau is from about 55 ksi to about 70 ksi.

In one embodiment, superelastic endoprostheses are configured to have a lower stress plateau that imparts increased fatigue resistance and increased resistance to the formation of cracks. Such an endoprosthesis can be configured to have a lower stress plateau of from about 5 ksi to about 50 ksi. In a preferred embodiment, the lower stress plateau is from about 20 ksi to about 40 ksi. In a more preferred embodiment, the upper stress plateau is from about 25 ksi to about 30 ksi.

Anisotropy is a measurement of a physical characteristic that has two different values in substantially orthogonal directions. For example, if all of the longitudinally aligned connectors of an endoprosthesis are more flexible than the substantially annular rings of the endoprosthesis, that endoprosthesis has an anisotropy in its stress measurement in the radial and longitudinal directions. The same physical measurement varies dependent upon which direction it is measured.

In one embodiment, endoprostheses are configured to have an anisotropic measurement of stress in the radial and longitudinal directions. The endoprosthesis may be configured to be radially stiff and longitudinally flexible, through using the heating methods described herein.

In one embodiment, an endoprosthesis may have an anisotropism imparted upon the area where the endoprosthesis makes contact with an intraluminal lesion by selectively imparting an increased radial stiffness to the area of the endoprosthesis that makes contact with the lesion area of the lumen. In this embodiment of the present invention, the superelastic endoprosthesis has an increased radial strength and less flexibility only at the part of the endoprosthesis where it is needed, at the lesion contact area. The rest of the endoprosthesis has an increased longitudinal flexibility, relative to the radial stiffness, and therefore still maintains the desirable flexibility for an intraluminal endoprosthesis.

Therefore, superelastic endoprostheses of the present invention are configured to have improved fatigue resistance over other endoprostheses through selectively raising the austenitic finish temperature of a given superelastic metal and/or superelastic endoprosthesis for a particular application. This can include superelastic endoprostheses where the whole body, majority of the body, minority of the body, or selected portion of the body have the austenitic finish temperature, upper stress plateau, and lower stress plateau characteristics as described herein.

III. Method of Manufacturing Endoprostheses

Designing a superelastic endoprosthesis is generally a four step process. The first step is to quantify the strains of the in vivo environment in which the endoprosthesis will be placed. The second step is to develop computational models and tests to be applied to a given endoprosthesis within a particular in vivo environment. The third step is to determine the maximum allowable mean and alternating strains given its in vivo application. In step four, the calculated strains from step two are combined with the allowable strains from step three to determine whether a design of a given endoprosthesis will be fatigue resistant enough for the given number of stress-strain cycles that the endoprosthesis will experience in its in vivo environment.

Once a design for a particular endoprosthesis is configured according to substantially the steps above, the endoprosthesis must then be manufactured. In general, a superelastic endoprosthesis may be manufactured through heating a superelastic material in the shape of a lasercut tube. If the superelastic endoprosthesis is required for a particular application, such as inside of a superficial femoral artery, the endoprosthesis may need to be as large as about 10 millimeters. Increasing the inner diameter of the superelastic nitinol hollow tube is a gradual process with individual steps often being expansions of only 1 millimeter. The number of steps that are repeated can be up to and over 10 steps. Therefore, in the case of having a superelastic nitinol hollow tube with an inner diameter of 1.3 millimeters, after about 8 steps, the diameter of the superelastic nitinol hollow tube may be around 10 millimeters, which is sufficient for most intraluminal endoprosthestic applications within the human body. The beginning diameter and the final diameter of the superelastic lasercut hollow tube may be smaller or larger depending upon the desired final diameter that an endoprosthesis needs to be for a particular application.

Additionally, self-expanding endoprosthesis composed of nitinol tubing have a pattern cut into the tubing using a laser, etching. Generally, a nitinol tube having an outer diameter, which is smaller than the expanded diameter, is used as the basis of the endoprosthesis. After cutting the pattern, the cut tube will be expanded and heat set to its desired diameter. After cutting or etching the nitinol tubing, it passed through additional steps, such as honing, bead blasting and electropolishing. The nitinol tubing may have a pattern cut into the tubing before or after the manufacturing steps herein.

When the superelastic endoprosthesis is configured to have the final diameter necessary for its particular application, the stent may then be heated according to the method of the present invention in order to configure the superelastic endoprosthesis to impart an austenitic finish temperature.

In one embodiment, a nickel titanium or nitinol self expanding endoprosthesis can be heat set. Heat setting is a process whereby the nitinol, or other superelastic material, is heated to a temperature far above its austenitic finish temperature, followed by water quenching. A superelastic endoprosthesis may be deformed at the heat set temperature into a new shape. When the endoprosthesis is cooled so it is in the martensitic form, the endoprosthesis may be deformed into a variety of shapes. When the deformed, martensitic endoprosthesis is introduced into a body lumen, for example, the temperature of the endoprosthesis rises to (and above) the austenitic finish temperature of the endoprosthesis and the endoprosthesis will then reform to the heat set shape.

The austenitic final temperature is configured by heating the superelastic endoprosthesis at a temperature far above its austenitic final temperature. This heating can also be conducted to heat set the endoprosthesis. In one embodiment, endoprostheses of the present invention are heated at a temperature of from about 400 degrees Celsius to about 600 degrees Celsius. In a preferred embodiment, endoprostheses of the present invention are heated at a temperature of from about 450 degrees Celsius to about 575 degrees Celsius. In a more preferred embodiment, the endoprostheses of the present invention are heated at a temperature from about 500 degrees Celsius to about 550 degrees Celsius.

The austenitic final temperature is configured by heating the superelastic endoprosthesis at a temperature far above its austenitic final temperature for a given time period greater than about 30 seconds. In one embodiment, endoprostheses of the present invention are heated from about 30 seconds to about 12 hours or longer. In a preferred embodiment, endoprostheses of the present invention are heated from about 40 seconds to about 1 hour. In a more preferred embodiment, the endoprostheses of the present invention are heated from about 1 minute to about 2 minutes.

The temperature and the time of heating of the superelastic endoprosthesis depend upon the composition of the superelastic metal and the particular application of the superelastic endoprosthesis. For example, a nitinol superelastic metal alloy having a composition of 49% nickel and 51% titanium can have different characteristics than a nitinol superelastic metal alloy having a binary composition of nickel and titanium. Using a standard superelastic nitinol (55.3-56.3 wt. % Ni), a temperature of about 500 degrees Celsius for about 30 seconds or more is preferable to configure the superelastic nitinol endoprosthesis to have an increased austenitic finish temperature. A useable range of temperatures for standard superelastic nitinol metals is from about 400 to about 600 degrees Celsius for greater than about 30 seconds. The temperature and time of exposure change according to the amount of increase of austenitic finish temperature that is needed. Temperature ranges and times of heat treatment also change when strengthening elements such as Cr are added.

After the endoprosthesis is heat treated to its desired diameter, i.e. heat set, additional heat treating can be applied to change the endoprosthesis' austenitic final temperature. The additional heat treatment temperature can range from about 350 degrees Celsius to about 550 degrees Celsius. In a preferred embodiment, the additional heat treatment ranges from about 400 to about 500 degrees Celsius. In a more preferred embodiment, the additional heat treatment ranges from about 425 to about 475 degrees Celsius. The duration of the additional heat treatment can range from about 30 seconds to about 30 minutes, and can be repeated multiple times until the target austenitic temperature is achieved. In a preferred embodiment, the duration of the additional heat treatment is from about 1 minute to about 15 minutes. In a more preferred embodiment, the duration of the additional heat treatment is from about 5 minutes to about 10 minutes.

In one embodiment, the superelastic endoprosthesis may be manufactured to selectively impart anisotropism between the longitudinal stiffness and the radial stiffness. The superelastic endoprosthesis may be configured to have a longitudinal stiffness that is more flexible compared to the radial stiffness. This anisotropism is imparted upon the superelastic endoprosthesis through selectively point heating a portion of the superelastic endoprosthesis, including the substantially annular elements or the connectors. The selective heating of the substantially annular elements or the connectors may be achieved by using a narrowly focused laser, plasma, ion beam, photo beam, or electron beam to heat the endoprosthesis.

In one embodiment, endoprostheses having anisotropic measurements of stress in the radial and longitudinal directions may be manufactured by heating at least a portion of the endoprosthetic body in a fluid, such as air, salt bath, or fluidized sand, having a temperature range and time of heating herein. The endoprosthetic body may be in the form of an endoprosthesis having substantially annular elements connected together by connectors. The entire body, a majority of the body, or only the connector or annular portions of the superelastic endoprosthesis may have the superelastic characteristics as described herein, depending upon which portions are heat treated.

### EXAMPLES

The following examples of methods for increasing the austenitic finish temperature of a superelastic endoprosthesis are exemplary and explanatory only and are not to be viewed as being restrictive of the invention.

### Example 1

The autstenitic finish temperature was modulated in accordance with the present invention by heating an endoprosthesis as described in Methods A-D. In Method A, a superelastic tube was heated in a plurality of heating cycles to modulate the austenitic finish temperature by the following: a superelastic tubing having an inner diameter of 2 mm is heat treated at 500 degrees Celsius for 2 minutes and slidably disposed upon a mandrel having a diameter of 3 mm, then the superelastic tubing is quenched in water; the tubing is then heat treated at 500 degrees Celsius for 2 minutes and slidably disposed upon a mandrel having a diameter of 4 mm, then the superelastic tubing is quenched in water; the tubing is then heat treated at 500 degrees Celsius for 2 minutes and slidably disposed upon a mandrel having a diameter of 5 mm, then the superelastic tubing is quenched in water; and the tubing is then heat treated at 500 degrees Celsius for 4 minutes and slidably disposed upon a mandrel having a diameter of 6 mm, then the superelastic tubing is quenched in water, having been heat set at a diameter of 6 mm.

In Method B, a superelastic tube was heated in a plurality of heating cycles to modulate the austenitic finish temperature by the following: a superelastic tubing having an inner diameter of 2 mm, is heat treated at 500 degrees Celsius for 2 minutes and slidably disposed upon a mandrel having a diameter of 3 mm, then the superelastic tubing is quenched in water; the tubing is then heat treated at 500 degrees Celsius for 2 minutes and slidably disposed upon a mandrel having a diameter of 4 mm, then the superelastic tubing is quenched in water; the tubing is then heat treated at 500 degrees Celsius for 2 minutes and slidably disposed upon a mandrel having a diameter of 5 mm, then the superelastic tubing is quenched in water; the tubing is then heat treated at 500 degrees Celsius for 2 minutes and slidably disposed upon a mandrel having a diameter of 5.5 mm, then the superelastic tubing is quenched in water; and the tubing is then heat treated at 500 degrees Celsius for 4 minutes and slidably disposed upon a mandrel having a diameter of 6 mm, then the superelastic tubing is quenched in water, having been heat set at a diameter of 6 mm.

In Method C, a superelastic tube was heated in a plurality of heating cycles to modulate the austenitic finish temperature by the following: a superelastic tubing having an inner diameter of 2 mm, is heat treated at 500 degrees Celsius for 2 minutes and slidably disposed upon a mandrel having a diameter of 3 mm, then the superelastic tubing is quenched in water; the tubing is then heat treated at 500 degrees Celsius for 2 minutes and slidably disposed upon a mandrel having a diameter of 4 mm, then the superelastic tubing is quenched in water; the tubing is then heat treated at 500 degrees Celsius for 2 minutes and slidably disposed upon a mandrel having a diameter of 4.5 mm, then the superelastic tubing is quenched in water; the tubing is then heat treated at 500 degrees Celsius for 2 minutes and slidably disposed upon a mandrel having a diameter of 5 mm, then the superelastic tubing is quenched in water; the tubing is then heat treated at 500 degrees Celsius for 2 minutes and slidably disposed upon a mandrel having a diameter of 5.5 mm, then the superelastic tubing is quenched in water; and the tubing is then heat treated at 500 degrees Celsius for 4 minutes and slidably disposed upon a mandrel having a diameter of 6 mm, then the superelastic tubing is quenched in water, having been heat set at a diameter of 6 mm.

In Method D, a superelastic tube was heated in a plurality of heating cycles to modulate the austenitic finish temperature by the following: a superelastic tubing having an inner diameter of 2 mm, is heat treated at 500 degrees Celsius for 2 minutes and slidably disposed upon a mandrel having a diameter of 3 mm, then the superelastic tubing is quenched in water; the tubing is then heat treated at 500 degrees Celsius for 2 minutes and slidably disposed upon a mandrel having a diameter of 4 mm, then the superelastic tubing is quenched in water; the tubing is then heat treated at 500 degrees Celsius for 2 minutes and slidably disposed upon a mandrel having a diameter of 5 mm, then the superelastic tubing is quenched in water; and the tubing is then heat treated at 500 degrees Celsius for 10 minutes and slidably disposed upon a mandrel having a diameter of 6 mm, then the superelastic tubing is quenched in water, having been heat set at a diameter of 6 mm.

The changes in austenitic finish temperature of the superelastic tubing from the heat set methods of A, B, C, and D are depicted in Table 1. Table 1 also depicts the change in austenitic finish temperature of the superelastic tubing after an additional heat treatment.

TABLE 1

| | | | | | | | Change |
|---|---|---|---|---|---|---|---|
| | | | | | | Af | of Af |
| | | | | Time | | (Celsius) | (Celsius) |
| | Af | | | (min.) of | Temperature | after | after |
| | (Celsius) | | Change | additional | (Celusius) of | additional | additional |
| | of | Af | of Af | heat | additional | heat | heat |
| | tubing | (Celsius) | (Celsius) | treatment | heat | treatment | treatment |
| | before | after | after | after heat | treatment | after heat | after heat |
| Heat set | heat set | heat set | heat set | set | after heat set | set | set |
| method | method | method | method | method | method | method | method |
| A | 8 | 24.5 | 16.5 | 15 | 450 | 36.7 | 12.2 |
| A | 8 | 24.4 | 16.4 | 15 | 400 | 31.1 | 6.7 |
| A | 8 | 28 | 20 | | | | |
| A | 8 | 25.5 | 17.5 | | | | |
| A | 8 | 26.5 | 18.5 | | | | |
| A | 8 | 27 | 19 | | | | |
| A | 8 | 24.2 | 16.2 | | | | |
| A | 8 | 23 | 15 | | | | |
| A | 8 | 22 | 14 | | | | |
| B | 8 | 20.0 | 12.0 | 10 | 450 | 36 | 16 |
| B | 8 | 20.2 | 12.2 | 10 | 450 | 36 | 15.8 |
| B | 3 | 22 | 19 | | | | |
| B | 3 | 22 | 19 | | | | |
| B | 3 | 26 | 23 | | | | |
| C | 8 | 21.2 | 13.2 | 10 | 450 | 36 | 14.8 |
| C | 8 | 22.5 | 14.5 | | | | |
| C | 8 | 23.5 | 15.5 | | | | |
| C | 3 | 24 | 21 | 10, three | 350 | 14.6 | −9.4 |
| | | | | times | | | |
| | | | | total | | | |
| C | 3 | 23 | 20 | 10 | 350 | 25 | 2 |
| Continued | | | | 10, two | 350 | 22 | −4 |
| from | | | | times | | | |
| above | | | | total | | | |
| C | 3 | 24.5 | 21.5 | 10, three | 350 | 16.1 | −8.4 |
| | | | | times | | | |
| | | | | total | | | |
| C | 3 | 23.5 | 20.5 | 10 | 350 | 26 | 2.5 |
| Continued | | | | 10, three | 350 | 22 | −4 |
| from | | | | times | | | |
| above | | | | total | | | |
| D | 8 | 22 | 14 | 5 | 450 | 29.7 | 7.7 |
| D | 8 | 23.6 | 15.6 | 5 | 450 | 30.9 | 7.3 |
| D | 8 | 24.5 | 16.5 | 5 | 450 | 31.6 | 7.1 |
| D | 3 | 25.5 | 22.5 | 5 | 450 | 29.1 | 3.6 |

### Example 2

In one embodiment, superelastic tubing is heat set and then subjected to an additional heat treatment for a duration ranging from 1 minute to 15 minutes and temperatures ranging from 350 degrees Celsius to 550 degrees Celsius. The resulting changes in austenitic finish temperatures of the superelastic tubing are compiled and depicted in Table 2.

TABLE 2

| Time of | Change in Af | Change in Af | Change in Af | Change in Af | Change in Af |
|---|---|---|---|---|---|
| additional | (in Celsius) | (in Celsius) | (in Celsius) | (in Celsius) | (in Celsius) |
| heat | after | after | after | after | after |
| treatment | additional | additional | additional | additional | additional |
| after heat | heat treatment | heat treatment | heat treatment | heat treatment | heat treatment |
| set | at 350 Celsius | at 400 Celsius | at 450 Celsius | at 500 Celsius | at 550 Celsius |
| 1 min. | −2 | | −1, 1, 6 | −1 | 2, 7 |
| 5 min. | −2 | | 2, 6.8, 7.7, | 1, 4.5 | |
| | | | 7.3, 7.1, 3.6 | | |
| 10 min. | −3, −3.4, −3.1, | | 9, 4.9, 16, | 3, 12.9 | |
| | −2.4, 2, 2.5, −3.1 | | 15.8, 14.8 | | |
| | 3.1, −2.8, −2.3, | | | | |
| | −2 | | | | |
| 15 min. | 6 | 6.7 | 5, 15.7, 12.2 | 8, −0.6 | |

The present invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. An endoprosthesis having improved fatigue resistance, the endoprosthesis comprising:
an endoprosthetic body comprised of a superelastic metal, where at least a portion of the superelastic metal is characterized by at least one of the following:
an austenitic finish temperature from about 5 to about 37 degrees Celsius;
a stress-strain curve of the superelastic metal having an upper plateau stress from about 40 ksi to about 80 ksi; or
a stress-strain curve of the superelastic metal having a lower plateau stress from about 5 ksi to about 50 ksi.

2. An endoprosthesis as in claim 1, wherein the superelastic metal is characterized by at least one of the following:
an austenitic finish temperature from about 30 to about 35 degrees Celsius;
a stress-strain curve of the superelastic metal having an upper plateau stress from about 50 ksi to about 70 ksi; or
a stress-strain curve of the superelastic metal having a lower plateau stress from about 20 ksi to about 40 ksi.

3. An endoprosthesis as in claim 1, wherein the superelastic metal has an austenitic finish temperature from about 10 to about 30 degrees Celsius.

4. An endoprosthesis as in claim 1, wherein the superelastic metal is comprised of a nickel-titanium alloy.

5. An endoprosthesis as in claim 1, wherein the superelastic metal is a nitinol.

6. An endoprosthesis as in claim 1, wherein the endoprosthetic body is a stent.

7. An endoprosthesis as in claim 1, wherein the endoprosthetic body is comprised of a plurality of substantially annular elements connected together with at least one connector, said connector having the superelastic metal characteristic.

8. An endoprosthesis as in claim 1, wherein at least a majority of the endoprosthetic body has the superelastic metal characteristic.

9. An endoprosthesis as in claim 1, wherein the superelastic metal characteristic is achieved by the following:
fabricating the endoprosthetic body;
heating at least a portion of the endoprosthetic body to a temperature from about 400 degrees Celsius to about 600 degrees Celsius; and
maintaining the temperature for at least about 30 seconds.

10. An endoprosthesis as in claim 9, wherein the heating is conducted by immersing at least a majority of the endoprosthetic body in a fluid having the temperature.

11. An endoprosthetic as in claim 10, wherein the fluid is a salt bath or fluidized sand.

12. An endoprosthesis as in claim 9, wherein the heating is conducted by point heating the portion of the superelastic metal.

13. An endoprosthesis as in claim 12, wherein the point heating is conducted by at least one of a laser, plasma, ion beam, photo beam, or electron beam.

14. A method of fabricating an endoprosthesis having improved fatigue resistance, the method comprising:
fabricating an endoprosthetic body comprised of a superelastic metal;
heating at least a portion of the superelastic metal to a temperature from about 400 degrees Celsius to about 600 degrees Celsius;
maintaining the temperature for at least about 30 seconds; and
configuring the superelastic metal to have at least one of the following characteristics:
an austenitic finish temperature from about 5 to about 37 degrees Celsius;
a stress-strain curve of the superelastic metal having an upper plateau stress from about 40 ksi to about 80 ksi; or
a stress-strain curve of the superelastic metal having a lower plateau stress from about 5 ksi to about 50 ksi.

15. A method as in claim 14, further comprising configuring the superelastic metal to have at least one of the following characteristics:
an austenitic finish temperature from about 30 to about 35 degrees Celsius;
a stress-strain curve of the superelastic metal having an upper plateau stress from about 50 ksi to about 70 ksi; or
a stress-strain curve of the superelastic metal having a lower plateau stress from about 20 ksi to about 40 ksi.

16. A method as in claim 14, further comprising configuring the superelastic metal to have an austenitic finish temperature from about 10 to about 30 degrees Celsius;

17. A method as in claim 14, wherein the heating is conducted by immersing at least a majority of the endoprosthetic body in a fluid having the temperature.

18. A method as in claim 17, wherein the fluid is a salt bath or fluidized sand.

19. A method as in claim 14, wherein the heating is conducted by point heating the portion of the superelastic metal.

20. A method as in claim 19, wherein the point heating is conducted by at least one of a laser, plasma, ion beam, photo beam, or electron beam.

21. A method as in claim 14, wherein the endoprosthetic body is comprised of a plurality of substantially annular elements connected together with at least one connector, said connector having the superelastic metal characteristic.

22. A method as in claim 14, wherein at least a majority of the endoprosthetic body has the superelastic metal characteristic.

23. A method as in claim 14, wherein the endoprosthetic body has been heat set to a desired diameter before the heating to achieve the austenitic finish temperature.

24. A method as in claim 23, wherein the heat set is achieved by at least one cycle of heating at least a portion of the superelastic metal to a temperature from about 350 degrees Celsius to about 550 degrees Celsius.

25. A method of selectively imparting an anisotropism to an endoprosthesis having improved fatigue resistance, the method comprising:
fabricating an endoprosthetic body comprised of a superelastic metal;
spot heating at least a portion of the superelastic metal to a temperature from about 400 degrees Celsius to about 600 degrees Celsius;
maintaining the temperature for at least about 30 seconds; and
configuring the spot heated portion of the superelastic metal to have at least one of the following characteristics:
an austenitic finish temperature from about 5 to about 37 degrees Celsius;
a stress-strain curve of the superelastic metal having an upper plateau stress from about 45 ksi to about 80 ksi; or
a stress-strain curve of the superelastic metal having a lower plateau stress from about 15 ksi to about 40 ksi.

26. A method as in claim 25, further comprising configuring the spot heated portion of the superelastic metal to have at least one of the following characteristics:
an austenitic finish temperature from about 15 to about 20 degrees Celsius;
a stress-strain curve of the superelastic metal having an upper plateau stress from about 60 ksi to about 80 ksi; or
a stress-strain curve of the superelastic metal having a lower plateau stress from about 20 ksi to about 40 ksi.

27. A method as in claim 25, further comprising configuring the spot heated portion of the superelastic metal to have an austenitic finish temperature from about 30 to about 35 degrees Celsius.

28. A method as in claim 25, configuring the endoprosthetic body to have an anisotropism between the longitudinal stiffness and the radial stiffness.

29. A method as in claim 28, wherein the longitudinal stiffness is more flexible compared to the radial stiffness.

30. A method as in claim 29, wherein the endoprosthetic body is comprised of a plurality of substantially annular elements connected together with at least one connector, said connector being more flexible compared to at least one of the substantially annular elements.

31. A method as in claim 25, wherein the spot heating is conducted by at least one of a laser, plasma, ion beam, photo beam, or electron beam.

32. A method as in claim 25, wherein the endoprosthetic body has been heat set to a desired diameter before the heating to achieve the austenitic finish temperature.

33. A method as in claim 32, wherein the heat set is achieved by at least one cycle of heating at least a portion of the superelastic metal to a temperature from about 350 degrees Celsius to about 550 degrees Celsius.
